(19) United States
(12) Patent Application Publication
(10) Pub. No.: US 2010/0145433 A1
(43) Pub. Date: Jun. 10, 2010

(54) ENDOPROSTHESES FOR DEPLOYMENT IN A BODY LUMEN

(75) Inventors: Boris Anukhin, San Jose, CA (US); Keif Fitzgerald, San Jose, CA (US)

Correspondence Address: WORKMAN NYDEGGER, 1000 EAGLE GATE TOWER,, 60 EAST SOUTH TEMPLE, SALT LAKE CITY, UT, 84111, US

(73) Assignee: Abbott Cardiovascular Systems, Inc., Santa Clara, CA (US)

(21) Appl. No.: 12/571,198

(22) Filed: Sep. 30, 2009

Related U.S. Application Data
(60) Provisional application No. 61/101,576, filed on Sep. 30, 2008.

(51) Int. Cl. A61F 2/82

(52) U.S. Cl. 623/1.16; 623/1.15

(57) ABSTRACT

Example embodiments include an endoprosthesis that has a first annular segment that is radially expandable and a second annular segment that is also radially expandable. An axial segment, which includes one or more struts, is operatively associated with the first annular segment and the second annular segment to maintain a specified distance between the first annular segment and the second annular segment.

### CROSS REFERENCE

This application claims the benefit of, and priority to, U.S. Provisional Patent Application Ser. No. 61/101,576, filed on Sep. 30, 2008 and entitled “ENDOPROSTHESES FOR DEPLOYMENT IN A BODY LUMEN,” which is incorporated in its entirety herein by this reference.

### BACKGROUND

1. The Field of the Invention

The present disclosure relates to various implantable medical devices deliverable and deployable within a lumen. More particularly, the invention relates to various vascular endoprostheses.

2. The Relevant Technology

Stents, grafts, and a variety of other endoprostheses are used in interventional procedures, such as for treating aneurysms, lining or repairing vessel walls, filtering or controlling fluid flow, and expanding or scaffolding occluded or collapsed vessels. Such endoprostheses may be delivered and used in virtually any accessible body lumen of a human or animal, and may be deployed by any of a variety of recognized means. One recognized use for a vascular endoprosthesis is for the treatment of atherosclerotic stenosis in blood vessels. For example, after a patient undergoes a percutaneous transluminal coronary angioplasty, or similar interventional procedure, a stent is often deployed at the treatment site to improve the results of the medical procedure and reduce the likelihood of restenosis.

To reduce the likelihood of restenosis, the stent may be configured to scaffold or support the treated blood vessel. If desired, the stent may also be loaded with a beneficial agent so as to act as a delivery platform to reduce restenosis or the like. Other suitable examples of medical conditions for which endoprostheses are an appropriate treatment include, but are not limited to, arterial aneurysms, venous aneurysms, coronary artery disease, peripheral artery disease, peripheral venous disease, chronic limb ischemia, blockage or occlusion of the bile duct, esophageal disease or blockage, defects or disease of the colon, tracheal disease or defect, blockage of the large bronchi, blockage or occlusion of the ureter, or blockage or occlusion of the urethra.

Typically, a vascular endoprosthesis, such as a stent, is delivered by a delivery sheath, such as a catheter, to a desired location or deployment site inside a body lumen or other tubular organ. The intended deployment site may be difficult to access by a physician and often involves moving the delivery system through a tortuous luminal pathway that may involve various turns or curves. Thus, to allow advancement through the luminal pathway to the deployment site, a vascular endoprosthesis may need to flex or otherwise bend to traverse the various curves.

When flexing or bending during delivery to the deployment site, large axial or radial forces may be exerted on the vascular endoprosthesis. Furthermore, once deployed, the vascular endoprosthesis may continue to experience large forces. For example, a vascular endoprosthesis deployed in a Superficial Femoral Artery (SFA) application undergoes longitudinal, bending, torsional, tensile and radial cyclical loading that may lead to fatigue failures of the vascular endoprosthesis. In particular, when the vascular endoprosthesis is forced to bend after being deployed, the portion of the vascular endoprosthesis on the outside radius of the bend may be forced to lengthen, while portions of the vascular endoprosthesis on the inside radius of the bend may be forced to shorten.

Due to the fact that the vascular endoprosthesis may not expand evenly, the lengthening and shortening of the vascular endoprosthesis generally increases fatigue failures. Current vascular endoprosthesis configurations that are subjected to these forces often fail. Failure may result in crack formation and possible stent fracture. In the event of stent fracture, the sharp edges may damage the vessel wall. Consequently, the fractured stent may cause thrombus formation or blockage within the vessel.

### BRIEF SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Embodiments disclosed herein relate to implantable medical devices and in particular to stents with good flexibility while providing good vessel coverage and radial force.

In one example embodiment, an endoprosthesis has a first annular segment that is radially expandable and a second annular segment that is also radially expandable. An axial segment, which includes one or more struts, is operatively associated with the first annular segment and the second annular segment to maintain a specified distance between the first annular segment and the second annular segment.

In another example embodiment, an endoprosthesis includes a first annular portion oriented about a central axis and a second annular portion oriented about the central axis. The endoprosthesis further includes a connection portion that has an axial segment with a first end and a second end. The first end of the axial segment is connected to the first annular portion and the second end of the axial segment is connected to the second annular portion.

In a further embodiment, an endoprosthesis includes a first column segment and a second column segment. The first and second column segments include a plurality of undulating column struts. The endoprosthesis further includes a column interface that couples the first column segment to the second column segment. Other embodiments may include additional column segments that are coupled with additional column interfaces.

Additional features and advantages of the disclosure will be set forth in the description which follows, and in part will be obvious from the description, or may be learned by the practice of the invention. The features and advantages of the invention may be realized and obtained by means of the instruments and combinations particularly pointed out in the appended claims. These and other features of the present invention will become more fully apparent from the following description and appended claims, or may be learned by the practice of the invention as set forth hereinafter.

### BRIEF DESCRIPTION OF THE DRAWINGS

To further clarify at least some of the advantages and features of the present disclosure, a more particular description will be rendered by reference to specific embodiments thereof, which are illustrated in the appended drawings. It is appreciated that these drawings depict only illustrated embodiments and are therefore not to be considered limiting of its scope. The disclosure will be described and explained with additional specificity and detail through the use of the accompanying drawings in which:

FIG. 1A illustrates a flat view of an example endoprosthesis;

FIG. 1B illustrates a perspective view of the example endoprosthesis illustrated in FIG. 1A;

FIG. 1C illustrates an example endoprosthesis illustrated in FIG. 1A in a flexed position;

FIG. 1D illustrates a flat view of a further example endoprosthesis;

FIG. 1E illustrates a perspective view of the example endoprosthesis of FIG. 1D;

FIG. 2A illustrates a flat view of an example endoprosthesis;

FIG. 2B illustrates a perspective view of the example endoprosthesis illustrated in FIG. 2A;

FIG. 3A illustrates a flat view of an example endoprosthesis;

FIG. 3B illustrates a perspective view of the example endoprosthesis illustrated in FIG. 3A; and

FIG. 4 illustrates an exemplary subject for an endoprosthesis

### DETAILED DESCRIPTION

In general, the present invention relates to an implantable medical device that is deliverable and deployable within a body lumen. More particularly, embodiments of the invention relate to an implantable medical device that is configured to withstand longitudinal, bending, torsional, tensional and radial loading without fracturing or otherwise compromising the structural integrity of the implantable medical device. Thus, example embodiments of the invention relate to implantable medical devices that, relative to medical devices lacking the same or similar configurations, can better withstand the loading conditions due to radial, axial, and torsional strains that exist during delivery of the implantable medical device, as well as the loading conditions that exist on the implantable medical device after deployment.

### I. Endoprostheses

One example of an implantable medical device is an endoprosthesis. The endoprosthesis may have various configurations that improve functionality and durability over an endoprosthesis that lacks the same or similar configurations. There are several factors that contribute to the overall functionality and durability of an endoprosthesis, including, but not limited to, axial flexibility, radial force (i.e., the amount of force an endoprosthesis may withstand before collapsing or otherwise failing structurally), size of cross-sectional profile in a delivery configuration, and scaffolding ability (i.e., the ability to reduce unsupported surface area (“USA”) of the body lumen.) Although the following description generally refers to stents, the present disclosure is not limited to stents, but rather may be used in any application that may benefit from the use of an endoprosthesis.

Embodiments of the invention include endoprosthesis or stents with portions that are oriented in various directions. Portions oriented in a circumferential direction can reduce the stress associated with bending. In other words, these portions allow expansion and contraction in an axial direction. The configuration of these circumferential portions can be optimized to distribute the axial force along the entire portion.

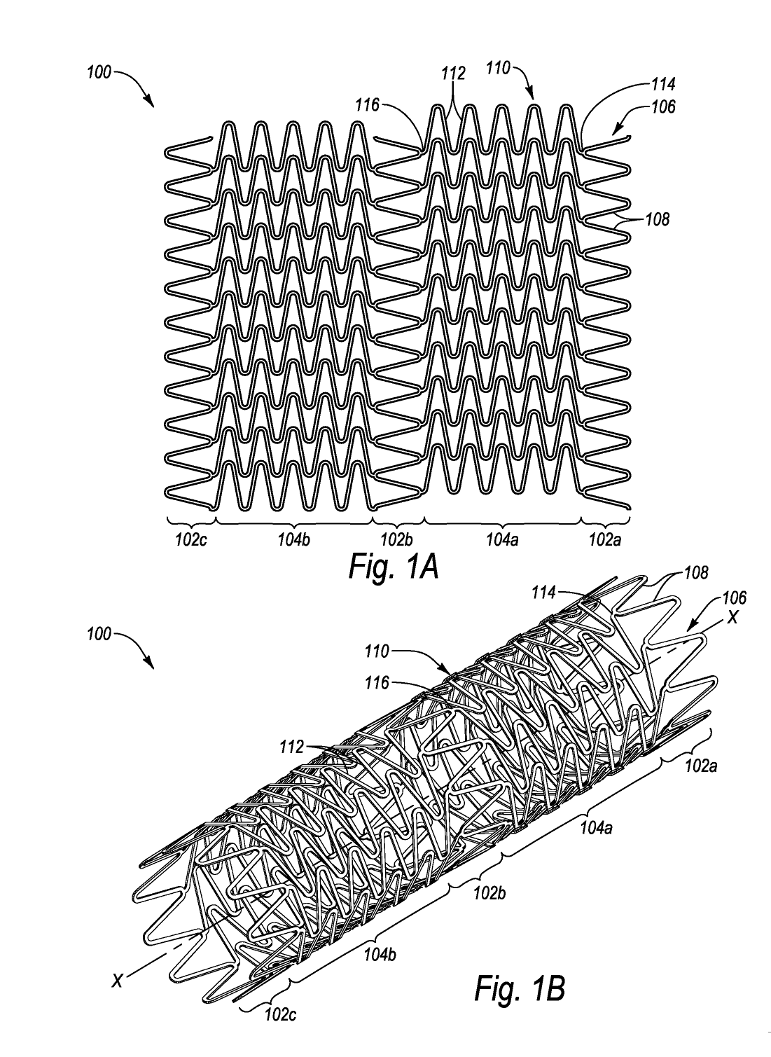

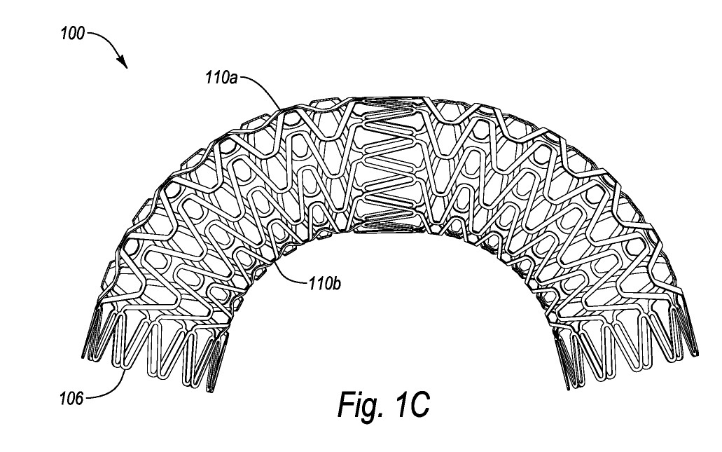

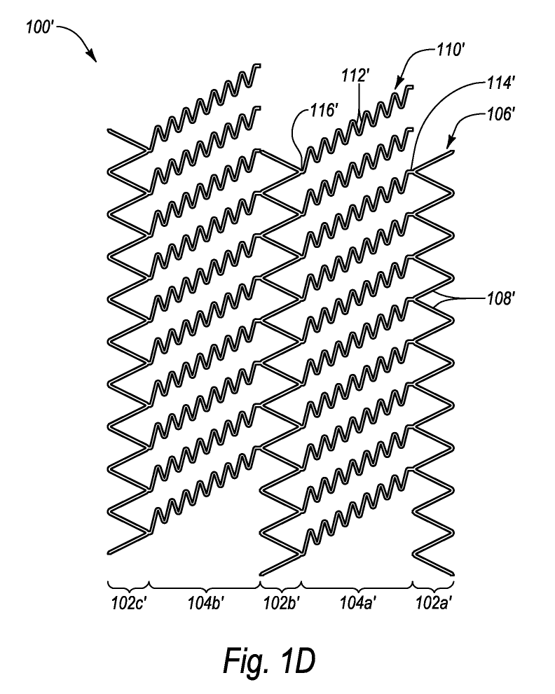

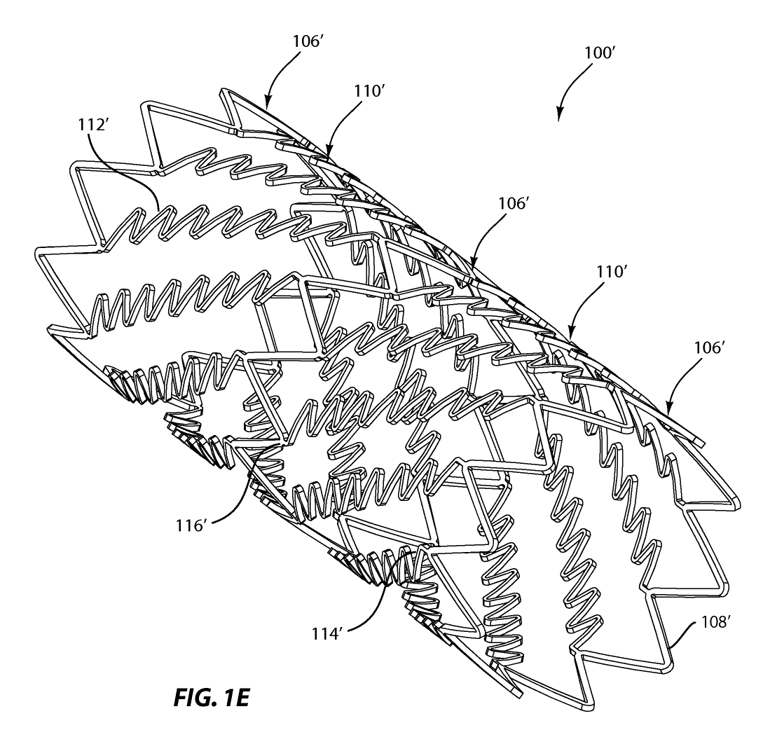

For example, FIGS. 1A and 1B illustrate one structural embodiment of an endoprosthesis 100 configuration. FIG. 1A is a flat view of the endoprosthesis 100 shown in FIG. 1B. FIGS. 1A and 1B illustrate features and configurations of the endoprosthesis 100.

The endoprosthesis 100 may include a first portion 102a and a second portion 104a. The first portion 102a may include one or more annular segments 106 that have an annular configuration that may be oriented generally about a central axis X, as shown in FIG. 1B. The annular segment 106 may include a plurality of annular struts 108 that may assist in forming the annular configuration around the central axis X, the annular struts 108 oriented generally in an axial direction, i.e., the annular struts have an orientation that is generally parallel to the central axis X, as shown in FIG. 1B. The second portion 104a of the endoprosthesis 100 may include one or more axial segments 110 positioned generally about the circumference of the stent. The axial segment 110 may include a plurality of axial struts 112 that may extend from a first end 114 toward a second end 116, the axial struts oriented in a circumferential direction, i.e., oriented generally perpendicular to the central axis, as shown in FIG. 1B. Adjacent annular segments 106 and axial segments 110 may be coupled or otherwise connected together at various interfaces between the annular segments 106 and the axial segments 110.

Generally, in operation, the endoprosthesis 100 may be configured to move from a pre-deployed state (typically compressed) toward a deployed state (typically expanded). While in the pre-deployed state, the endoprosthesis 100 is introduced into a body lumen, for example, a catheter or similar device may be used to introduce the endoprosthesis 100 into the body lumen. After introduction, and while still in the pre-deployed state, the endoprosthesis 100 may be maneuvered through the body lumen toward a deployment site. For example, the catheter may be maneuvered through the body lumen path. The pre-deployed state of the endoprosthesis 100 facilitates the ability of the endoprosthesis 100 to flex around the curves that may exist within the body lumen pathway. Upon reaching the deployment site, the endoprosthesis 100 may change from the pre-deployment state to the deployed state. For example, the endoprosthesis 100 may automatically expand to the deployed state upon being released from the catheter. Likewise, a balloon or similar device may be used to affect the transformation of the endoprosthesis 100 from the pre-deployed state to the deployed state.

Returning to FIGS. 1A and 1B, various aspects and configurations of the endoprosthesis 100 will be discussed in more detail. As mentioned above, the endoprosthesis 100 may include first portion 102a. In one example embodiment, the endoprosthesis may include more than one portion that has the same configuration as the first portion 102a. For example, and as illustrated in FIGS. 1A and 1B, the endoprosthesis 100 may include two additional portions 102b and 102c that have a configuration similar to the first portion 102a. In other embodiments, the endoprosthesis 100 may include only a single portion with a first portion 102a configuration, or alternatively, the endoprosthesis 100 may include more than three portions that have the first portion 102a configuration.

In addition to the number of portions that have the first portion 102a configuration, the number of portions with the second portion 104a configuration may also vary. In one embodiment, illustrated in FIGS. 1A and 1B, the endoprosthesis 100 may include an additional portion 104b that has the second portion 104a configuration. In other embodiments, however, the endoprosthesis may have more or less than two portions with the second portion 104a configuration.

Related to the number of portions of the endoprosthesis 100 that have either the first portion 102a or second portion 104a configuration is the arrangement of the portions with the first portion 102a configuration with respect to the portions with the second portion 104a configuration. In one embodiment, for example, sections with the first portion 102a configuration may alternate with sections with the second portion 104a configuration. For example, as illustrated in FIGS. 1A and 1B, portions 102a, 102b and 102c alternate with portions 104a and 104b. In other example embodiments, the portions with the first portion 102a configuration and the portions with the second portion 104a configuration may have various other arrangements.

As illustrated in FIGS. 1A and 1B, the first portion 102a includes a single annular segment 106. Alternatively, the first portion 102a may include a plurality of annular segments 106. With the addition of multiple annular segments 106 to the first portion 102a, the properties of the first portion 102a are maintained over a longer length of the endoprosthesis 100.

Just as with the number of annular segments 106 in the first portion 102a, the number of axial segments 110 within the second portion 104a may vary from one embodiment to the next. For example, and as illustrated in FIG. 1A, the second portion 104a includes axial segments 110 positioned at each peak of the annular segment 106. In other embodiments, the number of axial segments 110 may be greater or fewer depending on the overall configuration of the endoprosthesis 100.

In addition to the number of axial segments 110, the properties of the second portion 104a may be applied over a longer or shorter length of the endoprosthesis by adjusting the length of the axial segment(s) 110. Specifically, the axial segments 110 may be used to maintain an approximate specified spacing, or a range of spacing, between the annular segments 106. Using axial segments to maintain spacing between annular segments 106 may increase surface area coverage and improve scaffolding.

One way to maintain the second portion 104a properties is to lengthen the overall length of the second portion 104a. For example, FIG. 1A illustrates two portions 104a and 104b that have substantially the same length. However, in other embodiments, the lengths of the portions with the second portion 104a configuration may vary and be almost any length that provides ample scaffolding of the body lumen wall. Moreover, the lengths of the portions with the second portion 104a configuration may vary from one portion to the next within the same endoprosthesis embodiment. For example, the length of second portion 104a may vary from the portion 104b.

In addition to the length of the axial segments 110, the overall configuration of the axial segments 110, as well as the annular segments 106, may also vary from one embodiment to the next, and within the same embodiment. As mentioned, the annular segments 106 may include a plurality of annular struts 108 that form the annular segment 106. For example, and as illustrated in FIGS. 1A and 1B, the annular struts 108 may be configured as a series of sine waves, i.e., a wave formation that undulates from one strut 108 to the next. In other examples, the struts 108 may take various other geometric arrangements such as a square waves, triangular waves, saw tooth waves, or other wave patterns or combinations thereof. Likewise, the axial struts 112 of the axial segments 110 may vary in a similar fashion.

In embodiments where the annular struts 108 in the annular segment 106 and the axial struts 112 in the axial segments 110 form a sinusoidal wave formation, the radial force of the endoprosthesis may be adjusted by varying the period and/or amplitude, along with other aspects, or combinations thereof, of the strut wave formation. The radial force is directed outwardly from the central axis X. As mentioned, by decreasing the period of the sinusoidal wave formation made by the annular struts 108 (i.e., increasing the frequency) the radial force of the endoprosthesis may be increased. Likewise, a decrease in the amplitude of the annular struts 108 of the annular segment 106 may increase the radial force within the segment.

Specifically, the radial force may be adjusted by varying the frequency of the undulating struts because as the frequency increases, the radial spring rate increases. Similarly, as the amplitude of the annular struts 108 decreases, the radial spring rate increases. Thus, by varying both the amplitude and the frequency of the sinusoidal wave pattern of the annular struts 108 of the annular segment 106, the radial force may be adjusted.

In addition to adjusting the radial force, the axial flexibility may be adjusted by varying the period or amplitude of the sinusoidal wave formation of the axial struts 112 of the axial segment 110. For example, the sinusoidal wave formation may vary in amplitude and period along the same segment 110, thus allowing bending axial forces to be distributed substantially along the entire strut. Moreover, in an embodiment where the second portion 104a includes a plurality of axial segments 110, the sinusoidal wave characteristics of each second portion segment 100 may vary one with another to produce the desired properties in the second portion 104a.

More specifically, FIG. 1C illustrates the endoprosthesis 100 in a bent position. The configuration of endoprosthesis 100 allows the endoprosthesis to expand or compress axially. For example, and as illustrated in FIG. 1C, axial segment 110a is located on the outer radius of the bend. Due to the axial segment 110a configuration, the axial segment 110a can expand and/or elongate to accommodate the bend. Likewise, axial segment 110b is located on the inter radius of the bend. Again, due to the axial segment 110b configuration, the axial segment 110b can compress to accommodate the bend. Thus, the overall flexibility, both radially and axially, of the endoprosthesis 100 is increased with this configuration.

Another way in which the properties and characteristics of the first and second portions 102a and 104a may be adjusted is by varying the cross-sectional dimension of the annular struts 108 and 112 in the first and second portions 102a and 104a, respectively. For example, a larger cross-sectional dimension generally will produce a higher radial force. In the same respect, by varying the cross-sectional geometric configuration, the functional properties of the first and second portions 102a and 104a may be varied. For instance, in one embodiment the cross-sectional geometric configuration of the annular struts 108 and/or axial struts 112 may be substantially circular. In other embodiments, the cross-sectional geometric configuration of the annular struts 108 and/or axial struts 112 may be rectangular, triangular, oval, or any other geometric configuration. The cross-sectional geometric configuration and cross-sectional dimension of the annular struts 108 and axial struts 112 may vary from one segment to the next, from one strut 108 to the next, or within the same strut 108. In further embodiments, the peaks and valleys (or bends) of the annular segments 106 and/or axial segments 120 between adjacent annular struts 108 and/or axial struts 112 may have smaller cross-sections than the elongate portions of the annular struts 108 and axial struts 112 in order to increase the flexibility of annular segment 106 or axial segment 110 and/or facilitate flexing and bending between the annular struts 108 and/or axial struts 112.

At least a portion of a plurality of annular struts 108 and/or axial struts 112 in the first and/or second portion annular segments 106 and 110 may be nested while in a pre-deployed configuration, thus reducing the overall cross-sectional dimension of the endoprosthesis 100 while in the pre-deployed configuration. For example, in embodiments where some annular struts 108 may be nested, a peak or other portion of the annular strut 108 may be directed toward the central axis of the endoprosthesis 100 to reduce the cross-sectional dimension of the endoprosthesis 100 in the pre-deployed configuration. For instance, the peak of a one strut 108 may be positioned under another adjacent strut in the pre-deployed state. In further embodiments, the annular struts 108 and/or axial struts 112 may be shaped to accept and nest together with the annular struts 108 or axial struts 112 of an adjacent annular segment 106 or axial segment 110. For example, in some embodiments the annular struts 108 and/or axial struts 112 may include one or more bends consistent with respect to the shape of adjacent struts to allow for ease of nesting together with adjacent annular segments 106 or axial segments 110.

Due to the ability of the struts to nest, the overall surface area coverage of the endoprosthesis 100 may increase. For instance, because the struts may nest in the compressed state, the struts will be located closer to one another in the uncompressed state as compared to an endoprosthesis where the struts are not allowed to nest in the compressed state. Moreover, in one example embodiment, the endoprosthesis 100 may included nested struts in the uncompressed state, thus further increasing the surface area coverage, and likewise, improving the scaffolding of the endoprosthesis 100.

Although the term ‘central axis’ may generally connote a straight axis, the central axis of the endoprosthesis 100 may include any axis of any length that is oriented with respect to the endoprosthesis 100. For instance, if the endoprosthesis 100 is not straight along the entire length, the central axis may bend with respect to the non-straight portion of the endoprosthesis 100.

In order to form a structure around the central axis, the annular segment(s) 106 may be coupled to or otherwise attached to the axial segment(s) 110. For example, and as shown in FIGS. 1A and 1B, the axial segments 110 may couple to the annular segments 106 at the location of the annular segment 106 where two annular struts 108 meet, i.e., on the peak of the sinusoidal wave formation, as illustrated in FIG. 1A. In other embodiments, the axial segments 110 may couple to the annular segment 106 at any location along the annular segment 106, e.g., at any location along an annular strut 108. For example, the axial segment 110 may couple to the annular segment 106 at a location on the annular strut 108 between the peaks of the sinusoidal wave formation.

Just as the coupling location of the axial segment 110 to the annular segment 106 may vary, so too may the coupling pattern. For example, and as illustrated in FIGS. 1A and 1B, an axial segment 110 may be connected to every peak on the annular segment 106 that is adjacent to the second portion 104a. However, in other embodiments, the axial segment 110 may not be attached to every peak; rather, for example, the axial segment 110 may be attached to every other peak that is adjacent to the second portion 104a. In another embodiment, axial segment 110 may be attached to locations of the annular segment 106 between the peaks in addition to the axial segments 110 attached to the peaks of the annular segment 106.

Just as the coupling pattern may vary, the coupling angle of axial segments 110 to the annular segments 106 may also vary. For example, in some embodiments, an axial segment 110 may couple to the annular segment 106 at an angle of approximately 90° between the corresponding axial strut 112 and annular strut 108 at the coupling point. In further embodiments, the coupling angle may be approximately equal to or less than 45°. For example, in some embodiments, an axial segment 110 may couple to the elongate portion of an annular strut 108 of the annular segment 106 at an angle of approximately 30° or less between the annular strut 108 and the axial strut 112 at the coupling point.

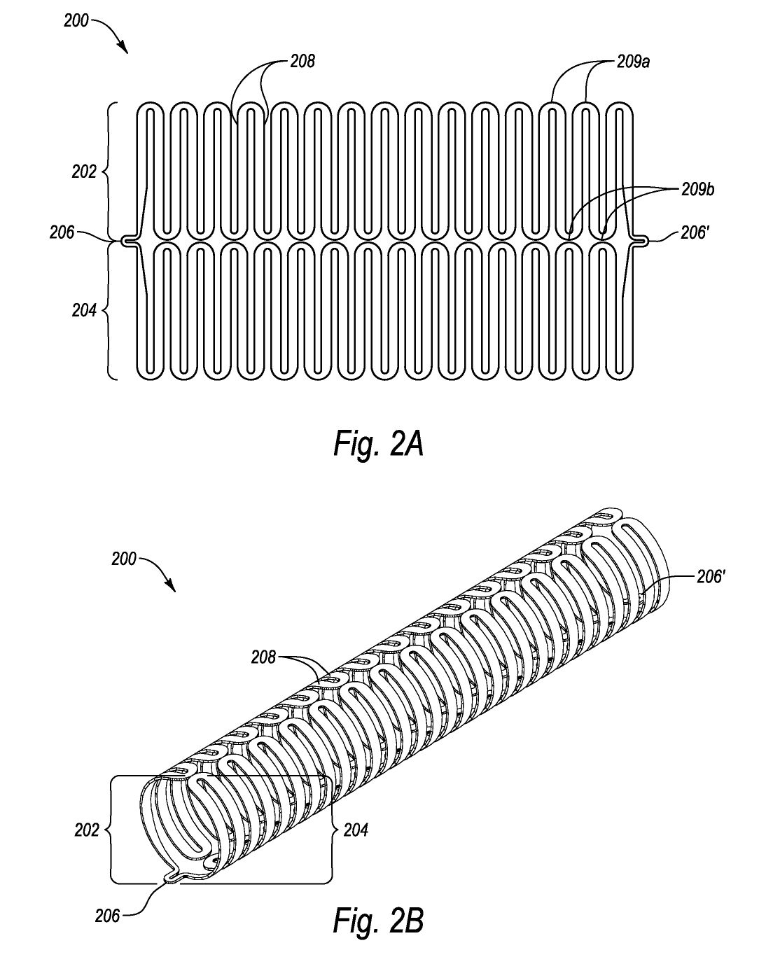

As shown in FIGS. 1A-1C, the axial segments 110 may be oriented substantially parallel with the central axis X of the endoprosthesis 100. In further embodiments, the axial segments 110 may vary in orientation with respect to the central axis X. For example, FIGS. 1D-1E illustrate one example endoprosthesis 100′ having axial segments 110′ with a varied orientation with respect to the central axis X. The endoprosthesis 100′ may be functionally similar to the endoprosthesis 100 previously described above and shown in FIGS. 1A-1C in most respects, wherein certain features will not be described in relation to this configuration wherein those components may function in the manner as described above and are hereby incorporated into the configuration described below. Like structures and/or components are given like reference numerals. Additionally, the endoprosthesis 100′ may incorporate at least one component of the endoprostheses 200, 300, and 410 described in connection with FIGS. 2-4, respectively.

In some embodiments, the axial segments 110′ may extend helically along the length of the endoprosthesis between corresponding annular segments 106′. For example, the axial segments 110′ may spiral around the central axis X as they extend along a length of the endoprosthesis 110. Moreover, the point of connection of the axial segments 110′ to a first annular segment 106′ may be rotationally offset from the point of connection of the axial segments 110′ to a second annular segment 106′ by one or more annular struts 108′. A manufacturer may vary the angle of spiral of the axial segments 110′ as desired for a particular application.

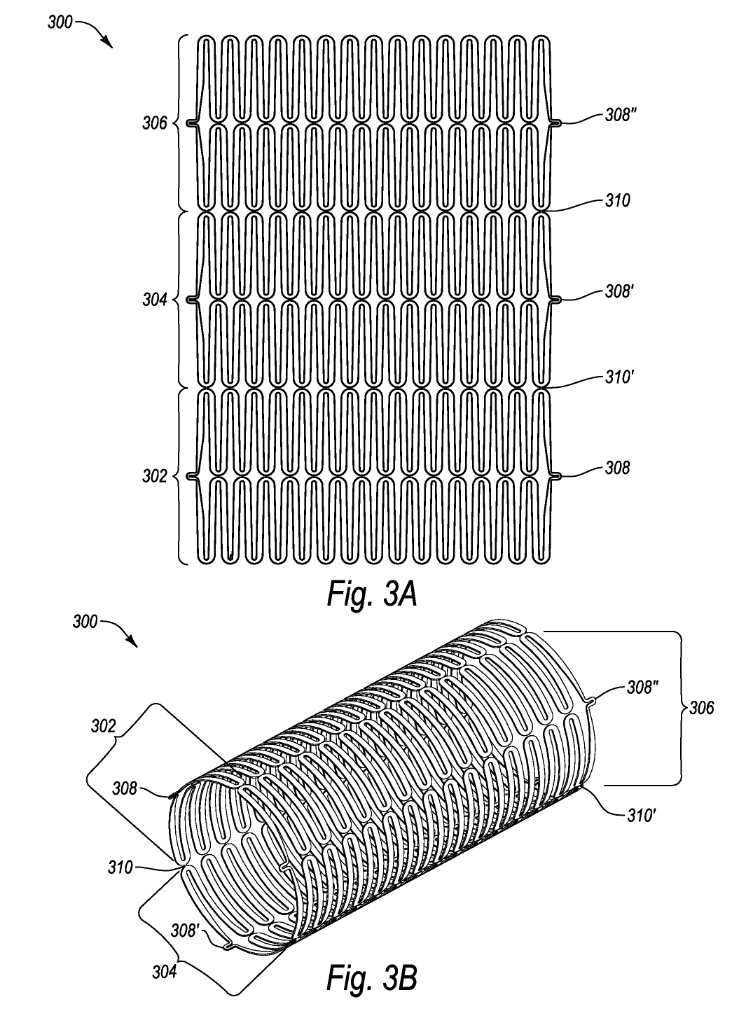

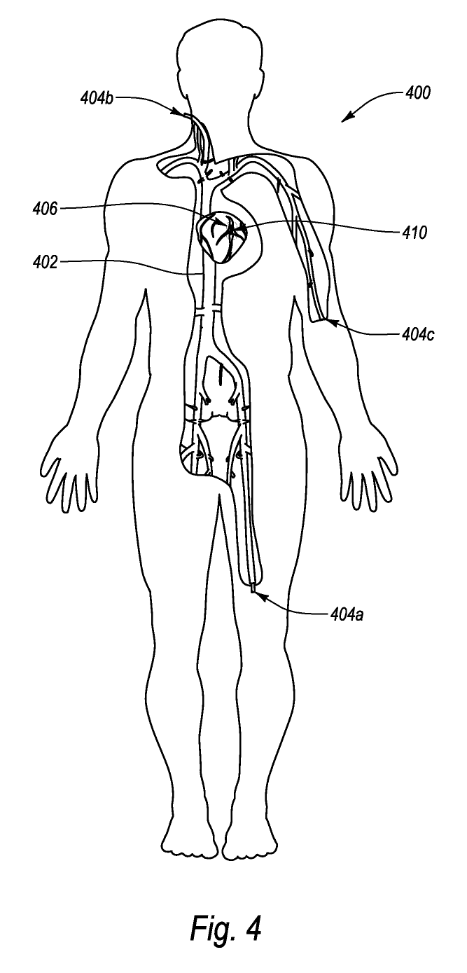

FIGS. 2A-2B illustrate a further example endoprosthesis 200 in accordance with the present disclosure. The endoprosthesis 200 may be functionally similar to the endoprostheses 100, 100′ previously described above and shown in FIGS. 1A-1E in most respects, wherein certain features will not be described in relation to this configuration wherein those components may function in the manner as described above and are hereby incorporated into the configuration described below. Like structures and/or components are given like reference numerals. Additionally, the endoprosthesis 200 may incorporate at least one component of the endoprostheses 300 and 410 described in connection with FIGS. 3-4, respectively.

The endoprosthesis 200, illustrated in FIGS. 2A and 2B, may include a first column segment 202 and a second column segment 204. The first and second column segments 202 and 204 may be connected at a column interface 206 and/or 206′, as indicated in FIG. 2A. Moreover, the first and second column segments 202 and 204 may be configured with a plurality of column struts 208.

The endoprosthesis 200 may be configured by forming the first and second column segments 202 and 204 into an annular configuration, as illustrated in FIG. 2B, for example. This configuration may provide high flexibility along with high radial force. Moreover, the profile of the endoprosthesis 500 in the pre-deployed configuration may be low compared to an endoprosthesis lacking a similar design.

As with the other previously discussed embodiments, the configuration of the first and second column segments 202 and 204 may vary. For example, the undulating pattern of the column struts 208, the amplitude and size, the number of column struts 208, the overall length and width of the column segment 202 and 204, and other physical and geometric configurations may also vary, as discussed above with reference to the previous embodiments. Moreover, the column segment 202 may be oriented in a spiral about the central axis. A spiral orientation, in particular, may provide increased flexibility.

After the endoprosthesis 200 is deployed, it may be easily collapsed by stretching or elongating the endoprosthesis 200. For example, in some embodiments, a user may collapse the endoprosthesis by applying a tensile force at the column interfaces 206 and 206′ to stretch the column segments 202 and 204 from their illustrated undulating configuration to a more straight and/or elongated configuration, Accordingly, a user can decrease the radial size of the endoprosthesis 200 in order to facilitate removal of the endoprosthesis 200 from a body lumen by stretching the column segments 202 and 204 from their shown undulating configurations to more elongated, straight, and/or collapsed configurations. In some embodiments, this may be facilitated by the lack of interconnections between adjacent struts 208 within a column segment 202 or 204. In further embodiments, the peaks 209a and valleys 209b in between adjacent struts 208 within a column segment 202 or 204 may have a reduced dimension and/or cross-section in order to increase flexibility of the column segments 202 and 204 at the peaks 209a and valleys 209b and thereby facilitate elongation of the column segments 202 and 204 into a more straight, collapsed configuration.

In addition, the endoprosthesis 200 may be deployed in a similar manner. For example, the endoprosthesis 200 may be initially disposed in a stretched, elongated, and/or collapsed configuration, such as within a delivery lumen. Once the endoprosthesis 200 is positioned for deployment, the user can deploy or release the endoprosthesis 200 from the delivery lumen. Thereafter, the endoprosthesis 200 may radially expand and/or shorten in length, whether mechanically or elastically, into the deployed configuration shown in FIG. 2B.

In some embodiments, the column struts 208 may be oriented substantially parallel with adjacent column struts 208, as illustrated in FIGS. 2A-2B. In further embodiments, the column struts 208 may be angled with respect to adjacent column struts, as illustrated in FIGS. 3A-3B. For example, in some embodiments, the column struts 208 may be positioned at approximately 45 degree angles with respect to adjacent column struts 208. In yet further embodiments, the column struts 208 may be positioned at angles greater than 45 degrees with respect to adjacent column struts 208. In even further embodiments, the column struts may be positioned at angles less than 45 degrees with respect to adjacent column struts 208. For example, this angle can range from 0 to 45 degrees in one embodiment, from 5 to 30 degrees in a further embodiment, and from 10 to 15 degrees in a yet further embodiment. In an additional example, this angle can range from 45 to 135 degrees in one embodiment, from 60 to 110 degrees in a further embodiment, and from 75 to 90 degrees in a yet further embodiment. In some embodiments, the angled configuration of the column struts 208 may facilitate elongation of the endoprosthesis 200.

FIGS. 3A and 3B illustrate a similar endoprosthesis 300. The endoprosthesis 300 may be functionally similar to the endoprostheses 100, 100′, and 200 previously described above and shown in FIGS. 1-2 in most respects, wherein certain features will not be described in relation to this configuration wherein those components may function in the manner as described above and are hereby incorporated into the configuration described below. Like structures and/or components are given like reference numerals. Additionally, the endoprosthesis 300 may incorporate at least one component of the endoprosthesis 410 described in connection with FIG. 4.

As in FIGS. 2A and 2B above, the endoprosthesis 300 includes a series of column segments. However, instead of just two column segments, as in FIGS. 2A and 2B, the endoprosthesis 300 may include a first column portion 302, a second column portion 304, and a third column portion 306. Each of the column portions 302, 304, and 306 may include column interfaces 308, 308′, and 308″ that couple or otherwise connect individual column segments. Moreover, each column portion 302, 304, and 306 may be connected to the adjacent column portion by way of a connector segment 310. For example, column portion 302 may be coupled to column portion 304 by one or more connector segments 310′, while column portion 304 may be coupled to column portion 306 by one or more connector segments 310, as illustrated in FIGS. 3A and 3B. The column portions can be connected, by way of example, at proximal and distal ends of the endoprosthesis 300. In further embodiments, the column portions 302, 304, and 306 may be oriented in a spiral about the central axis. A spiral orientation, in particular, may provide increased flexibility.

As discussed in more detail above, the endoprosthesis 300 may be collapsed by elongating the column portions 302, 304, and 306. For example, by elongating the column portions 302, 304, and 306, a user may collapse the column portions 302, 304, and 306 from their undulating configuration into a more elongate, substantially straight configuration in order to facilitate delivery of the endoprosthesis 300 to or removal of the endoprosthesis 300 from a body lumen.

Aspects of the foregoing endoprostheses may include other endoprosthetic structures, such as interconnectors, bumpers, other structures, or combinations thereof. Examples of interconnectors, bumpers, and other structures are described in U.S. patent application Ser. No. 11/374,923, filed Mar. 13, 2006, and entitled “Segmented Endoprosthesis,” the disclosure of which is hereby incorporated by reference and attached as Exhibit B.

FIG. 4 illustrates an exemplary subject 400 for an endoprosthesis 410. The endoprosthesis 410 may be functionally similar to the endoprostheses 100, 200, and 300 previously described above and shown in FIGS. 1-3 in most respects, wherein certain features will not be described in relation to this configuration wherein those components may function in the manner as described above and are hereby incorporated into the configuration described below. Like structures and/or components are given like reference numerals.

The endoprosthesis 410 may be implanted in a body lumen 402 of the subject 400. The endoprosthesis 410 may be inserted and/or retrieved through an access site 404a, 404b, and/or 404c. In the present embodiment, the access site may include a femoral artery access site 404a, a jugular vein access site 404b, a radial vein access site 404c, femoral vein, brachial vein, brachial artery, other access sites, or combinations thereof. For instance, the endoprosthesis 410 may be inserted through the femoral artery access site 404a and retrieved through the jugular or radial vein access site 404b, 404c. In another example, the endoprosthesis 410 may be inserted through the jugular vein access site 404b and retrieved through the femoral artery or radial vein access site 404a, 404c. In a further example, the endoprosthesis 410 may be inserted through the radial vein access site 404c and retrieved through the femoral artery or jugular vein access site 404a, 404b.

The endoprosthesis 410 may be inserted and retrieved through the radial vein access site 404c. Additionally, the endoprosthesis 410 may be inserted and retrieved through the jugular vein access site 404b. Further, the endoprosthesis 410 may be inserted and retrieved through the femoral artery access site 404a.

The endoprosthesis 410 may be deployed near a deployment site 406. In the present embodiment, the deployment site 406 may include a location within the heart. In other embodiments, other deployment sites may be used. For example, the deployment site 406 may include all larger veins.

### II. Endoprosthetic Composition

The above disclosed examples of a vascular endoprosthesis may be made from a variety of materials, such as, but not limited to, those materials which are well known in the art of vascular endoprosthesis manufacturing. This may include, but is not limited to, a vascular endoprosthesis having a primary material for both the annular segments and the axial segments. Alternatively, the axial segments may be made from material different from the annular segments. Generally, the materials for the vascular endoprosthesis may be selected according to the structural performance and biological characteristics that are desired.

In one configuration, the axial segments and/or the annular segments may have multiple layers, with at least one layer being applied to a primary material. The multiple layers on the axial segments and/or the annular segments may be resiliently flexible materials or rigid and inflexible materials. For example, materials such as Ti3Al2.5V, Ti6Al4V, 3-2.5Ti, 6-4Ti and platinum may be particularly good choices for adhering to a flexible material, such as, but not limited to, Nitinol and providing good crack arresting properties. The use of resiliently flexible materials may provide shock-absorbing characteristics to the axial segments, and/or annular segments, which may also be beneficial for absorbing stress and strains, which may inhibit crack formation at high stress zones. Also, the multiple layers may be useful for applying radiopaque materials to the vascular endoprosthesis.

Self-expanding embodiments of a vascular endoprosthesis may include a material made from any of a variety of known suitable materials, such as a shaped memory material (“SMM”). For example, the SMM may be shaped in a manner that allows for restriction to induce a substantially tubular, linear orientation while within a delivery sheath, but may automatically retain the memory shape of the vascular endoprosthesis once deployed from the delivery sheath. SMMs have a shape memory effect in which they may be made to remember a particular shape. Once a shape has been remembered, the SMM may be bent out of shape or deformed and then returned to its original shape by unloading the material from strain or heating. Typically, SMMs may be shape memory alloys (“SMA”) comprised of metal alloys, or shape memory plastics (“SMP”) comprised of polymers.

Usually, an SMA may have any non-characteristic initial shape that may then be configured into a memory shape by heating the SMA and conforming the SMA into the desired memory shape. After the SMA is cooled, the desired memory shape may be retained. This allows for the SMA to be bent, straightened, compacted, and placed into various contortions by the application of requisite forces; however, after the forces are released, the SMA may be capable of returning to the memory shape. The main types of SMAs are as follows: copper-zinc-aluminium; copper-aluminium-nickel; nickel-titanium (“NiTi”) alloys known as nitinol; and cobalt-chromium-nickel alloys or cobalt-chromium-nickel-molybdenum alloys known as elgiloy alloys. The temperatures at which the SMA changes its crystallographic structure are characteristic of the alloy, and may be tuned by varying the elemental ratios.

In one example, the primary material of a vascular endoprosthesis may be a NiTi alloy that forms superelastic nitinol. In the present case, nitinol materials may be trained to remember a certain shape, straightened in a delivery sheath, such as a catheter, or other tube, and then released from the delivery sheath to return to its trained shape. Also, additional materials may be added to the nitinol depending on a desired characteristic.

An SMP is a shape-shifting plastic that may be fashioned into a vascular endoprosthesis in accordance with the present invention. It may be beneficial to include at least one layer of an SMA and at least one layer of an SMP to form a multilayered body; however, any appropriate combination of materials may be used to form a multilayered vascular endoprosthesis. When an SMP encounters a temperature above the lowest melting point of the individual polymers, the blend makes a transition to a rubbery state. The elastic modulus may change more than two orders of magnitude across the transition temperature (“Ttr”). As such, an SMP may be formed into a desired shape of a vascular endoprosthesis by heating it above the Ttr, fixing the SMP into the new shape, and cooling the material below Ttr. The SMP may then be arranged into a temporary shape by force, and then resume the memory shape once the force has been applied. Examples of SMPs include, but are not limited to, biodegradable polymers, such as oligo(ε-caprolactone)diol, oligo(ρ-dioxanone)diol, and non-biodegradable polymers such as, polynorborene, polyisoprene, styrene butadiene, polyurethane-based materials, vinyl acetate-polyester-based compounds, and others yet to be determined. As such, any SMP may be used in accordance with the present invention.

For example, VERIFLEX, the trademark for CRG's family of shape memory polymer resin systems, currently functions on thermal activation which may be customizable from −20° F. to 520° F., allowing for customization within the normal body temperature. This allows a vascular endoprosthesis having at least one layer comprised of VERIFLEX to be inserted into a delivery sheath. Once unrestrained by the delivery sheath, the body temperature may cause the vascular endoprosthesis to return to its functional shape. The axial segments and the struts in the coupling segments may be formed of different materials or be formed from a different and/or overlapping set of materials or alloys such that they respond to temperature differently. Thus, the coupling segments may disengage or decouple during deployment without impacting the shape memory of the struts or of the coupling segments.

A vascular endoprosthesis having at least one layer made of an SMM or suitable superelastic material and other suitable layers may be compressed or restrained in its delivery configuration within a delivery device using a sheath or similar restraint, and then deployed to its desired configuration at a deployment site by removal of the restraint as is known in the art. A vascular endoprosthesis made of a thermally-sensitive material may be deployed by exposure of the vascular endoprosthesis to a sufficient temperature to facilitate expansion as is known in the art.

Balloon-expandable vascular endoprosthesis embodiments may be comprised of a variety of known suitable deformable materials, including stainless steel, silver, platinum, tantalum, palladium, cobalt-chromium alloys or other known biocompatible materials.

For delivery, the balloon-expandable vascular endoprosthesis having suitable materials may be mounted in the delivery configuration on a balloon or similar expandable member of a delivery device. Once properly positioned within the body lumen at a desired location, the expandable member may be expanded to expand the vascular endoprosthesis to its deployed configuration as is known in the art.

Also, balloon vascular endoprosthesis embodiments may include a suitable biocompatible polymer in addition to or in place of a suitable metal. The polymeric vascular endoprosthesis may include biodegradable or bioabsorbable materials, which may be either plastically deformable or capable of being set in the deployed configuration. If plastically deformable, the material may be selected to allow the vascular endoprosthesis to be expanded in a similar manner using an expandable member so as to have sufficient radial strength and scaffolding and also to minimize recoil once expanded. If the polymer is to be set in the deployed configuration, the expandable member may be provided with a heat source or infusion ports to provide the required catalyst to set or cure the polymer. Alternative known delivery devices and techniques for self-expanding endoprostheses likewise may be used.

Additionally, a self-expanding configuration of a vascular endoprosthesis may include a biocompatible material capable of expansion upon exposure to the environment within the body lumen. Examples of such biocompatible materials may include a suitable hydrogel, hydrophilic polymer, biodegradable polymers, bioabsorbable polymers. Examples of such polymers may include poly(alpha-hydroxy esters), polylactic acids, polylactides, poly-L-lactide, poly-DL-lactide, poly-L-lactide-co-DL-lactide, polyglycolic acids, polyglycolide, polylactic-co-glycolic acids, polyglycolide-co-lactide, polyglycolide-co-DL-lactide, polyglycolide-co-L-lactide, polyanhydrides, polyanhydride-co-imides, polyesters, polyorthoesters, polycaprolactones, polyesters, polyanydrides, polyphosphazenes, polyester amides, polyester urethanes, polycarbonates, polytrimethylene carbonates, polyglycolide-co-trimethylene carbonates, poly(PBA-carbonates), polyfumarates, polypropylene fumarate, poly(p-dioxanone), polyhydroxyalkanoates, polyamino acids, poly-L-tyrosines, poly(beta-hydroxybutyrate), polyhydroxybutyrate-hydroxyvaleric acids, combinations thereof, or the like. For example, a self-expandable vascular endoprosthesis may be delivered to the desired location in an isolated state, and then exposed to the aqueous environment of the body lumen to facilitate expansion.

Furthermore, the vascular endoprosthesis may be formed from a ceramic material. In one aspect, the ceramic may be a biocompatible ceramic which optionally may be porous. Examples of suitable ceramic materials include hydroxylapatite, mullite, crystalline oxides, non-crystalline oxides, carbides, nitrides, silicides, borides, phosphides, sulfides, tellurides, selenides, aluminum oxide, silicon oxide, titanium oxide, zirconium oxide, alumina-zirconia, silicon carbide, titanium carbide, titanium boride, aluminum nitride, silicon nitride, ferrites, iron sulfide, and the like. Optionally, the ceramic may be provided as sinterable particles that are sintered into the shape of a vascular endoprosthesis or layer thereof.

Moreover, the vascular endoprosthesis may include a radiopaque material to increase visibility during placement. Optionally, the radiopaque material may be a layer or coating on any portion of the vascular endoprosthesis. The radiopaque materials may be platinum, tungsten, silver, stainless steel, gold, tantalum, bismuth, barium sulfate, or a similar material.

### A. Biodegradable Coating Layers

It is further contemplated that the external surface and/or internal surface of the vascular endoprosthesis (e.g., exterior and luminal surfaces) may be coated with another material having a composition different from the primary endoprosthetic material. The use of a different material to coat the surfaces may be beneficial for imparting additional properties to the vascular endoprosthesis, such as providing radiopaque characteristics, drug-reservoirs, and improved biocompatibility.

In one configuration, the external and/or internal surfaces of a vascular endoprosthesis may be coated with a biocompatible material. Such coatings may include hydrogels, hydrophilic and/or hydrophobic compounds, and polypeptides, proteins or amino acids or the like. Specific examples may include polyethylene glycols, polyvinylpyrrolidone (“PVP”), polyvinylalcohol (“PVA”), parylene, heparin, phosphorylcholine, or the like. A coating material may include phosphorylcholine, as disclosed in U.S. Pat. No. 6,015,815 entitled “TETRAZOL-CONTAINING RAPAMYCIN ANALOGS WITH SHORTENED HALF-LIVES,” the entirety of which is herein incorporated by reference.

The coatings may also be provided on the vascular endoprosthesis to facilitate the loading or delivery of beneficial agents or drugs, such as therapeutic agents, pharmaceuticals and radiation therapies. As such, the endoprosthetic material and/or holes may be filled and/or coated with a biodegradable material.

Accordingly, the biodegradable material may contain a drug or beneficial agent to improve the use of the vascular endoprosthesis. Such drugs or beneficial agents may include antithrombotics, anticoagulants, antiplatelet agents, thrombolytics, antiproliferatives, anti-inflammatories, agents that inhibit hyperplasia, inhibitors of smooth muscle proliferation, antibiotics, growth factor inhibitors, or cell adhesion inhibitors, as well as antineoplastics, antimitotics, antifibrins, antioxidants, agents that promote endothelial cell recovery, antiallergic substances, radiopaque agents, viral vectors having beneficial genes, genes, siRNA, antisense compounds, oligionucleotides, cell permeation enhancers, and combinations thereof. Another example of a suitable beneficial agent is described in U.S. Pat. No. 6,015,815 and U.S. Pat. No. 6,329,386 entitled “TETRAZOLE-CONTAINING RAPAMYCIN ANALOGS WITH SHORTENED HALF-LIVES,” the entireties of which are herein incorporated by reference

In one configuration, the external surfaces of a vascular endoprosthesis may include a coating comprised of polytetrafluorethylene (“PTFE”), expanded PTFE (“ePTFE”), Dacron, woven materials, cut filaments, porous membranes, harvested vessels and/or arteries, or other such materials to form a stent graft prosthesis. Similarly, a medical device, such as a valve, a flow regulator or monitor device, may be used with the vascular endoprosthesis, such that the vascular endoprosthesis functions as an anchor for the medical device within the body lumen.

In one configuration, different external surfaces of a vascular endoprosthesis, such as a low stress zone less susceptible to flexing, may be coated with functional layers of an imaging compound or radiopaque material. The radiopaque material may be applied as a layer at low stress zones of the vascular endoprosthesis. Also, the radiopaque material may be encapsulated within a biocompatible or biodegradable polymer and used as a coating. For example, the suitable radiopaque material may be palladium platinum, tungsten, silver, stainless steel, gold, tantalum, bismuth, barium sulfate, or a similar material. The radiopaque material may be applied as layers on selected surfaces of the vascular endoprosthesis using any of a variety of well-known techniques, including cladding, bonding, adhesion, fusion, deposition or the like.

B. Matrix with Crack-Inhibiting Features

In addition to the foregoing compositions, a crack-inhibiting feature may be included within the material matrix of the vascular endoprosthesis. Exemplary crack-inhibiting features may include holes, fibers, particles, and bodies having multiple layers, such as planar layers or concentric layers. As such, any of the foregoing compositions may be impregnated and/or encapsulated with a suitable fibrous or particulate material. Also, a vascular endoprosthesis may be prepared to include a plurality of holes that extend through the endoprosthetic body. Moreover, the endoprosthetic body may have multiple layers separated by junctions or boundaries that inhibit crack propagation.

### III. Method of Making Endoprostheses

Various different manufacturing techniques are well known and may be used for fabrication of the segmented vascular endoprosthesis of the present invention. For example, the vascular endoprosthesis may be formed from a hollow tube using a known technique, such as laser cutting, EDM, milling, chemical etching, hydro-cutting, and the like. Also, the vascular endoprosthesis may be prepared to include multiple layers or coatings deposited through a cladding process such as vapor deposition, electroplating, spraying, or similar processes. Also, various other processes may be used such as those described below and or others known to those skilled in the art in light of the teaching contained herein.

Optionally, the vascular endoprosthesis may be fabricated from a sheet of suitable material, where the sheet is rolled or bent about a longitudinal axis into the desired tubular shape. Additionally, either before or after being rolled into a tube, the material may be shaped to include endoprosthetic elements by being shaped with well-known techniques such as laser-cutting, milling, etching or the like. If desired, the lateral edges of the structure may be joined together, such as by welding or bonding, to form a closed tubular structure, or the lateral edges may remain unattached to form a coiled, rolled sheet or open tubular structure. Such fabrication techniques are described in more detail below.

### A. Sintering

A method of making a vascular endoprosthesis in accordance with the present invention may include sintering sinterable particles to provide a sintered article having the shape of the vascular endoprosthesis. The sintering may be conducted in molds that are in the shape of a vascular endoprosthesis.

In one configuration, the sintered body may be obtained from a molded green body prepared by molding a mixture of sinterable particles with or without a binder into the shape of a vascular endoprosthesis or body intermediate. Sintering a molded green body that has the shape of a vascular endoprosthesis may provide a sintered body that may function as a vascular endoprosthesis with no or minimal additional processing. Alternatively, after the green body has been formed in the mold and sintered into a hardened vascular endoprosthesis, the process may include shaping the sintered body with a stream of energy and/or matter in order to obtain a desired shape. Thus, sintering a green body in a mold may result in a vascular endoprosthesis that is either ready for use, or requires additional processing or finishing.

Additionally, the sintered body may be shaped into a vascular endoprosthesis as described herein. Also, the vascular endoprosthesis may be further processed after sintering and/or shaping such as by grinding, sanding, or the like to provide enhanced surface characteristics.

### B. Drawing Concentric Tubes

In one configuration, a multilayered vascular endoprosthesis in accordance with the present invention may be prepared by a drawing process that draws two or more distinct concentric tubes into a single tube having two or more layers. Additionally, such a drawing process may combine multiple concentric tubes into a single multilayered tube. The drawing process may be configured to produce junctions separating adjacent layers or bonds that bond adjacent layers. As such, the sequentially-adjacent concentric tubes may be drawn together and progressively reduced in a cross-sectional profile until the desired size and residual clamping stress is attained.

Accordingly, a metallurgical bond may be prepared with elements of each sequentially-concentric tube diffusing together and bonding so as to form a strong metallurgical bond. Such a metallurgical bond may be achieved by applying significant pressure and heat to the tubes. As such, a metallurgical bond may form a diffusion layer at the interface between sequentially-adjacent concentric tubes (i.e., layers). The characteristics of these diffusion layers may be controlled by the proper heat treatment cycle. In part, this is because the heat treatment, temperature, and time of processing may control the rates of transfer of the diffusing elements that produce the diffusion layers. Also, the pressure at the interface between layers may be developed so as to result in the residual radial clamping stress in the tube after drawing.

In one example of this process, an outer tube of nitinol, a middle tube of tantalum, and an inner tube of Nitinol may be arranged to form the composite structure. The multilayered material may be produced to result in bonding between the layers so as to achieve a residual clamping stress of about 50 p.s.i. Accordingly, the annealing process may be performed within a limited range of time and temperatures. For example, the lower limit may be at least about 1550° F. for about six minutes, and the upper limit may be about 1850° F. for about 15 minutes.

In another configuration, a metallic interleaf layer may be placed between separate tubes so as to bond the tubes together and form a multilayered material. The multiple tubes separated by the metallic interleaf layer may be drawn together and progressively reduced until the desired cross-sectional profile and residual clamping stress is attained, as described above. The drawn tubes may be heat-treated to form a diffusion bond between the separate layers. As such, the metallic interleaf layer may enhance the diffusion rate or type of diffusing atoms that are transported across a diffusion region between one layer and the interleaf layer.

In one configuration, a multilayered sheet may be prepared to have separate layers of different materials or the same material. For example, the multilayered sheet may have a top layer of nitinol, a middle layer of tantalum, and a bottom layer of Nitinol. The sheet may be prepared by metallurgically bonding the layers prior to a deep drawing process, which is well known in the art. During the deep drawing process, the sheet may be placed over a die and forced into the die, such as by a punch or the like. A tube having a closed end and a defined wall thickness may be formed in the die. This process may be repeated using a series of dies that have progressively decreasing diameters until a multilayered tube is formed having the desired diameter and wall thickness. For certain material combinations, intermediate heat treatments may be performed between the progressive drawing operations to form a multilayered material that is resistant to delaminating. Once a multilayered tube of desired thickness and dimensions has been formed, the closed end and the curved edges may be cut off. Then, the tube may be heat treated, as described above, until proper inter-metallic bonds are formed between the layers.

### C. Shaping

Accordingly, an endoprosthetic material may be shaped by various methods as described in more detail below. Such shaping techniques may utilize streams of energy and/or streams of matter in order to impart shapes into the endoprosthetic material. The streams of energy include photons, electromagnetic radiation, atomic, and sub-atomic materials, as described above. On the other hand, the streams of matter are considered to include materials larger than atomic scale particles, and may be microscopic or macroscopic in size. In any event, the shaping may be designed to direct a stream of energy or a stream of matter at the endoprosthetic material to form an endoprosthetic element and/or holes therein.

In one configuration, a stream of energy may cut, shape, and/or form a tube into an endoprostheses by generating heat at the site where the stream intersects the material, as is well known in the art. The thermal interaction may elevate the local temperature to a point, which may cut, melt, shape, and/or vaporize portions of the endoprosthetic material from the rest of the material.

Accordingly, one configuration of the stream-cutting apparatus may operate and shape the endoprosthetic material by thermal interactions. As such, any of the thermal processes described herein may be used for thermal-cutting. For example, such thermal interactions may arise from laser beam treatment, laser beam machining, electron beam machining, electrical discharge machining, ion beam machining, and plasma beam machining.

In one configuration, by knowing the thermal properties of the endoprosthetic material, precise energy requirements may be calculated so that the thermal beam provides the appropriate or minimum energy for melting and/or vaporizing the material without significantly melting undesirable portions of the material. For example, laser beams are a common form of a stream of energy that may be used to shape the endoprosthetic material. Additionally, there are instances where a laser is preferred over all other cutting techniques because of the nature of the resulting vascular endoprosthesis as well as the characteristics of the endoprosthetic material.

In one configuration, a vascular endoprosthesis may be manufactured as described herein using a femtosecond laser. A femtosecond laser may be desirable in producing a vascular endoprosthesis in accordance with the multilayered composite structure of the present invention because it produces a smaller heat influence zone (“HIZ”) or heat affected zone (HAZ) compared to other lasers, or it may substantially eliminate the HIZ or HAZ. In comparison, cutting a vascular endoprosthesis using known methods may result in the tubular material being melted away, and thereby forming the pattern in the tubular member. Such melting may result in embrittlement of some materials due to oxygen uptake into the HIZ.

In one configuration, electrical discharge machining is used to shape endoprosthetic material and/or form holes in the endoprosthetic material as desired. As such, electrical discharge machining may be capable of cutting all types of conductive materials such as exotic metal including titanium, hastaloy, kovar, inconel, hard tool steels, carbides, and the like. In electrical discharge, the main interaction between the stream of energy and the endoprosthetic material is thermal, where heat is generated by producing electrical discharges. This may lead to the endoprosthetic material being removed by melting and evaporation. Some examples of electrical discharge machining include wire electron discharge machining, CNC-controlled electrical discharge machining, sinker electrical discharge machining, small hole discharge machining, and the like.

In another configuration, a charged particle beam may be used for shaping the endoprosthetic material, wherein electron beams and ion beams exemplify charged particle beams. A charged particle beam is a group of electrically-charged particles that have approximately the same kinetic energy and move in approximately the same direction. Usually, the kinetic energies are much higher than the thermal energies of similar particles at ordinary temperatures. The high kinetic energy and the directionality of these charged beams may be useful for cutting and shaping of the green bodies, as described herein. Additionally, there are some instances where electron beams or ion beams are preferred over other cutting techniques.

In one configuration, a stream of chemical matter may be used in order to shape or form holes in the endoprosthetic material. Chemical-jet milling, for example, provides selective and controlled material removal by jet and chemical action. As such, the process is similar to water-jet cutting, which is described in more detail below. In any event, chemical-jet milling may be useful for shaping various types of endoprosthetic materials, which provides intricate shaping capabilities.

In another configuration, electrochemical shaping may be based on a controlled electrochemical dissolution process similar to chemical-jet milling an endoprosthetic material. As such, the endoprosthetic material may be attached to an electrical source in order to allow an electrical current to assist in the shaping.

In one configuration, hydro-cutting or water-jet cutting may be used to shape an endoprosthetic material. Hydro-cutting is essentially a water-jet technology that uses the high force and high pressure of a stream of water directed at the endoprosthetic material in order to cut and shape the material as desired. Hydro-cutting may be preferred over some of the other stream-cutting technologies because it may be free of heat, flame, and chemical reactions, and may provide a precise cold shaping technique. Also, heated water with or without being doped with reactive chemicals may also be used. Hydro-cutting is particularly suitable for polymeric endoprostheses, but may be used for metal materials when combined with abrasive particles, as described below.

Additionally, hydro-cutting may be enhanced by the introduction of particulate materials into the water feed line. As such, some hydro-cutting techniques utilize garnet or other rigid and strong materials in order to apply an abrasive cutting force along with the force applied by the water itself. Also, the hydro-cutting process in the present invention may be used with or without inclusion of such abrasives.

Additionally, one of the benefits of hydro-cutting is the ability to reutilize and recycle the spent water-jet material. As such, the endoprosthetic material may be easily separated from the spent water, thereby enabling the recycling and reuse of the water during the hydro-cutting process.

In one configuration, sandblasting, which fits into the regime of stream of matter cutting, may be used to shape an endoprosthetic material by projecting a high energy stream of sand particles at the material. Sandblasting cuts materials in a manner similar to hydro-cutting, especially when the water-jet is doped with abrasive particulates. Additionally, various other particulate streams other than sand may be used in the stream-cutting techniques and machinery.

### D. Additional Processing

An additional step of passivation may be performed during the manufacturing stage of the vascular endoprosthesis in order to form a homogeneous oxide layer for corrosion-resistance. The passivation process may be performed prior to installation of the markers in accordance with the present invention or it may be performed after installation of the radiopaque markers. Alternatively, multiple passivation processes may be performed, once prior to application of the markers, and again after insertion of the markers.

As originally shaped and/or fabricated, the vascular endoprosthesis may correspond to its delivery configuration, to a deployed configuration, or to a configuration therebetween. The vascular endoprosthesis may be fabricated with a configuration at least slightly larger than the delivery configuration. In this manner, the vascular endoprosthesis may be crimped or otherwise compressed into its delivery configuration in a corresponding delivery device.

In another configuration, the vascular endoprosthesis may be originally fabricated from a tube having a diameter corresponding to the deployed configuration. In this manner, the longitudinally-free portions of the segments (e.g., elbow or foot not at a connection location) and circumferentially-free portions (e.g., the toe and/or heel portion of the foot extensions) may be maintained within the general cylindrical shape (e.g., diameter) of the vascular endoprosthesis when deployed, so as to avoid such portions from extending radially inward when in the deployed configuration. The vascular endoprosthesis may be designed to match the target vessel in which the vascular endoprosthesis is to be deployed. For example, a stent may be provided with an outer diameter in the deployed configuration ranging from about 1 mm for neurological vessels to about 25 mm for the aorta. Similarly, a stent may be provided with a length ranging from about 5 mm to about 200 mm. Variations of these dimensions will be understood in the art based upon the intended application or indication for the vascular endoprosthesis.

Also, the geometry of each component of the vascular endoprosthesis or endoprosthetic element, such as the width, thickness, length and shape of the strut elements, coupling elements, crossbars, connectors, elbows, foot portions, ankle portions, toe portions, heel portions and the like may be selected to obtain predetermined expansion, flexibility, foreshortening, coverage scaffolding, and cross-sectional profile characteristics. For example, longer crossbars and/or connectors may promote greater radial expansion or scaffolding coverage. The phase difference or circumferential alignment between adjacent segments likewise may be altered to control coverage and flexibility. Similarly, the number and placement of coupling locations and, if present, the axial segments, between longitudinally-adjacent segments may be selected to obtain the desired flexibility of the vascular endoprosthesis. The number of elbows and/or foot extensions between coupling locations also may be varied to achieve desired performance characteristics.

E. Coupling Adjacent Annular Elements and/or Sub-Endoprostheses

After the different annular elements and/or sub-endoprotheses, which can be formed by the same or different processes, are prepared, they are coupled into an endoprosthesis. The different annular elements and/or sub-endoprostheses can be coupled by any possible method, including methods of coupling different medical devices. For example, the different annular elements and/or sub-endoprostheses can be coupled into an endoprosthesis by brazing, forming a metallurgical bond, welding, forming a sleeve, or affixation with an adhesive. Other methods are also possible.

### IV. Method of Delivering Endoprosthesis

Generally, the endoprosthesis of the present disclosure can be delivered into a body of a subject. For example, the method of using catheters to deploy self-expandable or balloon-expandable stents can be employed.

In one embodiment, the endoprosthesis of the present disclosure are configured for use in a body lumen. As such, the present disclosure includes a method of delivering an endoprosthesis into a body lumen of a subject. Such a method includes: providing an endoprosthesis as described herein; orienting the endoprosthesis into a delivery orientation with a cross section that is smaller than the body lumen; inserting the endoprosthesis in the delivery orientation into a delivery device, such as a deliver catheter that can be configured substantially as a catheter for delivering a stent; delivering the endoprosthesis to a desired deployment site within the body lumen of the subject; removing the endoprosthesis from the delivery device; and expanding the endoprosthesis so as to have an enlarged dimension that applies radial forces to an inner wall of the body lumen.

In one embodiment, the present disclosure can include a method of extracting the endoprosthesis from the body lumen, which can include: inserting an endoprosthesis-extracting medical device into the body lumen so as to come into contact with the endoprosthesis; engaging the endoprosthesis-extracting medical device with the endoprosthesis; radially compressing the endoprosthesis so as to have a reduced dimension with a cross section that is smaller than the body lumen; and retrieving the endoprosthesis from the desired deployment site within the body lumen of the subject. Optionally, the endoprosthesis can be received into the endoprosthesis-extracting medical device, which can be substantially similar to a catheter.

In one embodiment, at least one of delivering or retrieving the endoprosthesis is performed with a catheter. Catheters configured for delivering and/or retrieving endoprostheses from a body lumen can be adapted for delivering and/or retrieving the endoprosthesis of the present disclosure.

The present disclosure may be embodied in other specific forms without departing from its spirit or essential characteristics. The described embodiments are to be considered in all respects only as illustrative and not restrictive. The scope of the disclosure is, therefore, indicated by the appended claims rather than by the foregoing description. All changes which come within the meaning and range of equivalency of the claims are to be embraced within their scope.

What is claimed is:

1. An endoprosthesis that is radially expandable, comprising:
a first portion including an annular segment that is orientated about a central axis; and
a second portion coupled to the first portion and including one or more axial segments extending along the central axis.

2. The endoprosthesis of claim 1, wherein the annular segment comprises a plurality of struts in an undulating wave configuration with corresponding peaks and valleys.

3. The endoprosthesis of claim 2, wherein the one or more axial segments each comprise a plurality of struts in an undulating wave configuration.

4. The endoprosthesis of claim 3, wherein the axial segments couple to the annular segments at the peaks of the undulating wave configuration.

5. The endoprosthesis of claim 1, further comprising a third portion that includes an annular segment, the second portion positioned between the first portion and the third portion.

6. The endoprosthesis of claim 1, wherein the second portion comprises a plurality of axial segments.

7. The endoprosthesis of claim 6, wherein the plurality of axial segments are oriented substantially parallel with the central axis.

8. The endoprosthesis of claim 6, wherein the plurality of axial segments extend helically about and along the central axis.

9. The endoprosthesis of claim 2, wherein the one or more axial segments couple to the plurality of struts of the first annular segment at the midpoints between the peaks and valleys.

10. The endoprosthesis of claim 5, wherein the second portion couples the first portion to the third portion, and wherein the axial segment of the second portion couples a peak of the first portion to a valley of the third portion.

11. An endoprosthesis comprising:
a first annular portion oriented about a central axis;
a second annular portion oriented about the central axis; and
a connection portion including one or more axial segments with a first end and a second end, the first end being connected to the first annular portion, the second end being connected to the second annular portion.

12. The endoprosthesis of claim 11, wherein the first annular portion and the second annular portion further comprise:
a plurality of struts having a non-linear configuration, wherein the plurality of struts undulate from one strut to the next in a wave configuration.

13. The endoprosthesis of claim 12, wherein the non-linear configuration of the plurality of struts comprises one or more bends in or in between otherwise linear struts.

14. An endoprosthesis comprising:
a first column segment including a first proximal end, a first distal end, and a first plurality of undulating column struts that extend from the first proximal end toward the first distal end of the first column segment;
a second column segment including a second proximal end, a second distal end, and a second plurality of undulating column struts that extend from the second proximal end toward the second distal end of the second column segment; and
a column interface that couples the first column segment to the second column segment.

15. The endoprosthesis of claim 14, wherein the first plurality of undulating column struts form a sinusoidal wave configuration.

16. The endoprosthesis of claim 15, wherein the second plurality of undulating column struts form a sinusoidal wave configuration.

17. The endoprosthesis of claim 16, wherein the endoprosthesis forms a substantially tubular member about a central axis in a deployed configuration.

18. The endoprosthesis of claim 17, wherein the column interface is located at the first and second proximal ends of the first and second column segments.

19. The endoprosthesis of claim 18, further comprising an additional column interface located at the first and second distal ends of the first and second column segments.

20. The endoprosthesis of claim 16 wherein one or more of the first plurality of column struts are substantially parallel with adjacent column struts, and one or more of the second plurality of column struts are substantially parallel with adjacent column struts.

21. The endoprosthesis of claim 16, wherein one or more of the first plurality of column struts are angled with respect to adjacent column struts and one of more of the second plurality of column struts is angled with respect to adjacent column struts.

22. The endoprosthesis of claim 16, where each column segment is configured to collapse to a substantially straight member upon elongation of the endoprosthesis.
